(19) United States
(12) Patent Application Publication
(10) Pub. No.: US 2026/0284329 A1
(43) Pub. Date: Sep. 24, 2026

(54) METHOD FOR DETERMINING AT LEAST ONE PARAMETER FOR THE VAPORIZATION IN AN INHALER, AND AN INHALER

(71) Applicant: KÖRBER TECHNOLOGIES GMBH, Hamburg (DE)

(72) Inventors: Frank GOLDSCHMIDTBÖING, Ortenberg (DE); Uwe PELZ, Nauenburg (DE); Muhannad GHANAM, Freiburg (DE); Armin JAMALI, Freiburg (DE); Eiko BÄUMKER, Freiburg (DE); Mohammadreza SABERI, Freiburg (DE); Peter WOIAS, Freiburg (DE); Thomas BILGER, Buggingen (DE); Jan JAKLIN, Eislingen (DE)

(21) Appl. No.: 19/641,792

(22) Filed: Apr. 8, 2026

Related U.S. Application Data
(63) Continuation of application No. 17/911,919, filed on Mar. 15, 2021.

(30) Foreign Application Priority Data
Mar. 16, 2020 (DE) 10 2020 107 091.8

(51) Int. Cl. A61M 11/04; A24F 40/57; A61M 15/06

(52) U.S. Cl. CPC A61M 11/042; A24F 40/57; A61M 15/06

(57) ABSTRACT

A method for determining at least one parameter for vaporization in an inhaler comprising a vaporizer, based upon resistive heating, and an electronic control device is characterized in that the electronic control device is configured to carry out the following initialization procedure-in particular, after a replacement of a vaporizer cartridge in the inhaler: outputting an inhalation request to a user of the inhaler; in the case of a puff taken by the user following the inhalation request, operating the vaporizer with a comparatively low vaporization capacity P, and recording a time measurement series of an electrical parameter of the vaporizer; determining a transition point ÜP between a region of low vaporization and a region of high vaporization in the recorded time measurement series; determining and storing at least one parameter associated with the initialization procedure.

### CROSS REFERENCE TO RELATED APPLICATIONS

This application is a Continuation Application of U.S. Ser. No. 17/911,919, filed Sep. 15, 2022; which is a National Stage Application of International Application Number PCT/EP2021/056495, filed Mar. 15, 2021; which claims priority to German Patent Application No. 10 2020 107 091.8, filed Mar. 16, 2020.

### FIELD OF THE INVENTION

The present invention relates to a method for determining at least one parameter for vaporization in an inhaler comprising a vaporizer, based upon resistance heating, and an electronic control device.

### BACKGROUND OF THE INVENTION

The temperature at the vaporizer is typically determined by means of a temperature-dependent electrical resistance of the vaporizer. The temperature of the vaporizer can be set in a targeted manner by the relationship between the temperature and the electrical resistance of the vaporizer. The temperature should not exceed a temperature determined by the liquid to be vaporized, since, otherwise, pollutants can arise—particularly if the vaporizer dries out.

The circuit of a vaporizer or heater can be described in simple terms as a series circuit of electrical resistances. Elements of this series circuit comprise an electrical resistance of the vaporizer (vaporizer resistance), a battery internal resistance, and unwanted parasitic electrical resistances. The parasitic resistances are provided, for example, in the form of the following resistances: an electrical resistance belonging to the electrical control device, a current-measuring resistance, an electrical resistance of the supply lines—in particular, through connecting wires, copper conductors, and/or soldering points—and, optionally, an electrical resistance of a possible plug connection. The parasitic resistance is neither temporally constant nor reproducible, since, for example, plug connections have an influence on the parasitic resistance that can be measured only with considerable effort, depending upon the state of aging, contamination, and/or deformation. Errors in temperature measurement due to parasitic resistances can lead to overheating of the liquid to be vaporized, which can lead to nucleate boiling or pollutant formation. Due to the various errors caused by measurement and parasitic currents, the vaporizer can be controlled only inadequately with known methods.

A temperature measurement by the change in resistance of the heating element or vaporizer is very imprecise under the following circumstances: if a vaporizer cartridge is connected to a base part of the inhaler via a detachable connector; if the heating element or the vaporizer has a low resistance in the ohm range and/or a low temperature coefficient. This is primarily due to the variation in the resistance of the connector due to varying contact pressure, contamination, and possibly corrosion.

As already described in German patent application no. 10 2019 113 645.8, it is advantageous for the vaporizer control in normal consumption operation to measure the resistance or, in the case of a known voltage, the current at which vaporization starts (transition point). This, advantageously, takes place online during regular vaporization, and thus in real time. Detection of the vaporization point (transition point) based upon the temporal profile of the resistance or current is difficult particularly if it is to take place in real time in the inhaler.

### BRIEF SUMMARY OF THE INVENTION

It is the object of the invention to provide a method with which parameters useful for vaporization can be reliably determined in an inhaler having limited data processing resources.

This object is achieved with the features of the independent claims.

According to the invention, the electronic control device is configured to perform the following initialization procedure-in particular, after a replacement of a vaporizer cartridge in the inhaler: outputting an inhalation request to a user of the inhaler; in the case of a puff taken by the user following the inhalation request, operating the vaporizer with a comparatively low vaporization capacity, and recording a time measurement series of an electrical parameter of the vaporizer; determining a transition point between a region of low vaporization and a region of high vaporization in the recorded time measurement series; determining and storing at least one parameter associated with the initialization procedure.

It could be shown that the transition point can be detected much better at low heating powers than at high heating powers, which are desirable in standard operation, since they enable rapid response and high vapor generation. The basic idea is therefore to initially carry out an initialization procedure for a cartridge once. For this purpose, the user—in particular, after inserting a vaporizer cartridge into the inhaler—is requested to take a puff. This puff is carried out at a comparatively low heating power (for example, set via pulse width modulation).

The vaporization or transition point is much easier to determine under these controlled conditions than with high-power, standard puffs. In addition, the calculation of the transition point may take longer than is desired during normal or consumption operation. In other words, the runtime of the initialization procedure is less critical than in normal consumption operation. Advantageously, the calculation of the (initialization) parameters can take longer than a normal consumption puff by the user—in particular, for example, longer than 1 s.

Advantageously, the heating power P set during the initialization procedure is not more than 80%, and, further advantageously, not more than 60%, of the average heating power <P> or maximum heating power Pmax used in normal consumption operation.

Preferably, one or more of the following parameters are determined and stored: the value R0 of the electrical parameter at the beginning of the time measurement series at ambient temperature T0; the ambient temperature T0; the time period tü from an initial point of the time measurement series until the transition point ÜP is reached; the value Rü of the electrical parameter at the transition point ÜP. In one variant of the initialization procedure, advantageously, the following are stored: the measured initial resistance R0 (that is the resistance of vaporizer plus detachable connector) at ambient temperature T0, the ambient temperature T0 itself, the time period tü until the transition point is reached, and the resistance value Rü at the transition point.

The initialization procedure can, advantageously, be carried out via a user puff. However, several puffs can also be analyzed, to increase accuracy. In general, the initialization procedure and/or the checking procedure can be carried out via one user puff or via a plurality of user puffs.

In an advantageous embodiment, the control device calculates a measure of the quality of the determination of the transition point, and, in case of insufficient quality, causes the initialization procedure to be carried out again.

Preferably, the heater of the vaporizer is controlled on the basis of the at least one stored parameter after completion of the initialization procedure. The vaporizer, in a subsequent consumption phase, is thus operated based upon the now stored model of the heater, i.e., the control thresholds are adapted to the measured data.

In an advantageous embodiment, the initialization procedure is carried out successively at different heating powers of the vaporizer, in order to be able to create a more accurate model of the heater.

Preferably, at least one of the determined and stored parameters is determined after a consumer puff in a checking procedure and is compared with a target value. This occurs-in particular, after completion of the initialization phase-in the consumption phase. In the case of deviations of the redetermined parameter from the target value, a predetermined measure is initiated-for example, performing a re-initialization procedure or outputting a message to the user.

Advantageously, all or some of the stored parameters (initial resistance at ambient temperature, the ambient temperature itself, the duration until the transition point is reached, and resistance value at the transition point) are thus determined after a puff in the consumption phase and compared to the internal model or target values derived therefrom. In the case of deviations from the expected target values, appropriate measures can be initiated, such as, for example, re-initialization or message to the user, e.g., in the form of “cartridge empty,” “check contact pressure and contamination of the connector,” “unexpected error, please contact support,” and the like. Such a checking procedure can take place every time at certain intervals or for certain events (interruption of use for a predetermined period of time, change in external temperature by a predetermined value, replacement of the cartridge, switching on of the inhaler, etc.).

In an advantageous embodiment, the initialization procedure and/or the checking procedure is thus carried out in an event-driven manner, i.e., after determination of at least one predetermined event, e.g., replacement of a vaporizer cartridge; switching on of the inhaler; interruption of use of the inhaler for a predetermined period of time; change in ambient temperature T0 by a predetermined value.

In an advantageous embodiment, an assessment—in particular, at least one subjective perception of the inhalation experience during the initialization procedure by the consumer—is requested in the initialization procedure, e.g., by input means on the inhaler or via a mobile communications device in communications connection with the inhaler. The requested assessment by the consumer can, advantageously, comprise one or more of the following categories: vapor quantity (for example, too high, too low, acceptable), taste (for example, too scratchy, too mild, acceptable), etc. Preferably, the requested assessment can be taken into account in the determination according to the invention of the at least one parameter.

In an advantageous embodiment, the initialization procedure and/or the checking procedure is carried out in a time-controlled manner, i.e., in each case at a specific time or on a certain day of the week, or periodically.

If the vaporizer cartridge has a readable identifier, after replacing the vaporizer cartridge and reinserting the vaporizer cartridge into the inhaler, it may be advantageous to go back to the already-stored model assigned to the identifier. If such an identifier does not exist or is not stored, an initialization is, advantageously, carried out during each replacement of the cartridge. In other words, when inserting a vaporizer cartridge with an individual identifier and having the identifier read out by the electronic control device, at least one stored parameter assigned to this identifier can be accessed by the electronic control device and used for vaporization control.

Preferably, the transition point ÜP is determined based upon a regression—preferably a linear regression—to the time measurement series. It has been shown, in particular, that the heating resistance at low heating powers changes linearly in time until the transition point is reached. Therefore, a simple method for determining the transition point is to adapt a straight line to the measuring points, starting from t0. The vaporization point is reached when the difference between the measured parameter or measured resistance and the straight line falls below or exceeds a preset threshold.

A method for determining the transition point can also be found using artificial intelligence methods. In this procedure, large data sets of exemplary vaporization curves are generated beforehand. After the recording, pre-processing can take place. This often comprises filtering, conversion into other forms of representation such as a frequency domain, and/or simpler mathematical operations such as integration or differential calculation. The results in the new and usually reduced feature space are then automatically or manually classified, in sections, into previously defined categories. These categories can, for example, be heating phase, vaporization phase, and cooling phase. For example, artificial neural networks or other tree structures can be used as classification methods. These classifiers can also be created by means of machine learning. The generated data offer, for example, the possibility of using monitored learning. The completed classifier can be programmed directly into the electronic control device after the learning/creation phase. Transition points can be recognized not only directly by a class, but also be determined, for example, by detecting the change of the detected class into another class in more or less real time. Such systems are known to be relatively robust against environmental fluctuations, as can arise due to scattering of the resistance as a result of deviations in the production process or the transition resistances. The accuracy of the system can be continuously improved by further analysis and recording of further measurement curves. Unmonitored and thus automatic learning would also be conceivable. For this purpose, the data required for this could be acquired directly by the inhaler and collected and used at a central location.

The object is, moreover, achieved by an inhaler comprising a vaporizer, based upon resistance heating, and an electronic control device which is configured or programmed to carry out the method described above.

### BRIEF DESCRIPTION OF THE DRAWINGS

The invention is explained below on the basis of preferred embodiments with reference to the accompanying drawings, in which:

FIG. 1 shows a schematic view of an electronic inhaler;

FIG. 2 shows a schematic circuit for the electrical heating of a heating element;

FIG. 3 shows a resistance-time diagram with four measurement series for different heating powers;

FIGS. 4A-4D show resistance-time diagrams, each with a measurement series from FIG. 3; and

FIG. 5 shows a 1/tü diagram against the heating power for four different heating powers.

### DETAILED DESCRIPTION OF THE INVENTION

The electronic inhaler 110—here, an electronic cigarette product—comprises a housing 11 in which an air channel 30 is provided between at least one air inlet opening 31 and one air outlet opening 24 on a mouth end 32 of the cigarette product 10. The mouth end 32 of the cigarette product 10 means the end at which the consumer puffs for inhalation, thus creating a negative pressure in the cigarette product 10 and an air flow 34 in the air channel 30.

The inhaler 10 advantageously consists of a base part 16 and an exchangeable vaporizer cartridge 17, which comprises a vaporization device 20 and a liquid reservoir 18. The air sucked in through the inlet opening 31 is guided in the air channel 30 as an air flow 34 to, through, or along the vaporization device 20. The vaporization device 20 is or can be connected to the liquid reservoir 18, in which at least one liquid 33 is stored. The vaporization device 20 vaporizes liquid 33, which is supplied to it from the liquid reservoir 18, and supplies the vaporized liquid as an aerosol/vapor into the air flow 34 at an outlet side 26. An advantageous volume of the liquid reservoir 18 is in the range between 0.1 mL and 5 mL, preferably between 0.5 mL and 3 mL, and more preferably between 0.7 mL and 2 mL or 1.5 mL.

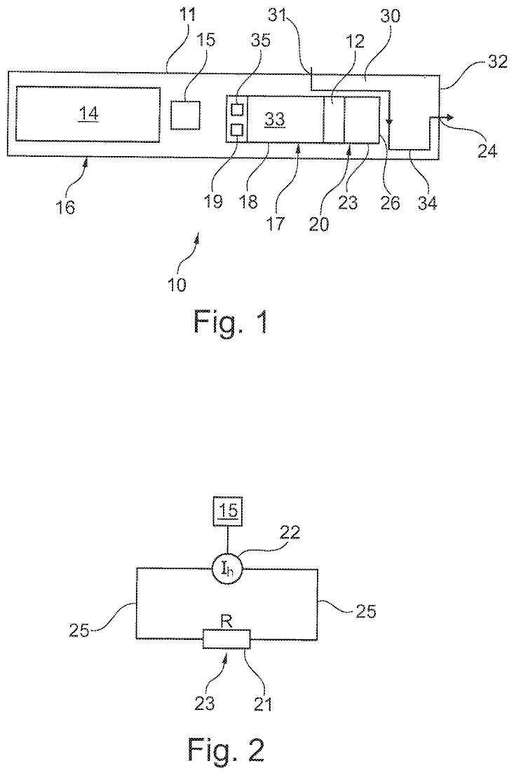

The electronic cigarette 10 further comprises an electrical energy store 14 and an electronic control device 15. The energy store 14 is generally arranged in the base part 16 and can, in particular, be an electrochemical disposable battery or a rechargeable electrochemical battery—for example, a lithium ion battery. In the example shown in FIG. 1, the energy store 14 is arranged in one part, facing away from the mouth end 32, of the inhaler 10. The vaporizer cartridge 17 is advantageously arranged between the energy store 14 and the mouth end 32. The electronic control device 15 comprises at least one digital data processing device—in particular, a microprocessor and/or microcontroller-in the base part 16 (as shown in FIG. 1) and/or in the vaporizer cartridge 17.

A sensor, e.g., a pressure sensor or a pressure or flow switch, is advantageously arranged in the housing 11, wherein the control device 15 can determine, based upon a sensor signal output by the sensor, that a consumer is puffing at the mouth end 32 of the inhaler 10 in order to inhale. In this case, the control device 15 controls the vaporization device 20 in order to add liquid 33 from the liquid reservoir 18 as aerosol/vapor into the air flow 34.

The liquid 33 which is stored in the liquid reservoir 18 and is to be dosed is, for example, a mixture comprising one or more of the following components: 1,2-propylene glycol, glycerol, water, at least one flavor, at least one active ingredient-for example, nicotine.

The vaporization device 20 comprises at least one vaporizer 23 with at least one resistance heating element 21 (see FIG. 2) and, advantageously, a wick element 12 for supplying liquid 33 from the liquid reservoir 18 to the vaporizer 23. When the vaporizer cartridge 17 is inserted in the inhaler 10, the resistance heating element 21 is electrically connected via electrical lines 25 to a heat current source 22 that is controllable by the electronic control device 15. The heat current source 22 draws electrical energy from the energy store 14. Due to the ohmic resistance, a current flow through the electrically-conductive heating element 21 leads to heating of the same and therefore to vaporization of liquid that is in contact with the heating element 21. Vapor/aerosol produced in this way escapes from the vaporizer 23 to the outlet side 26 and is admixed with the air flow 34; see FIG. 1.

The vaporization temperature is preferably in the range between 100° C. and 400° C., more preferably between 150° C. and 350° C., and even more preferably between 190° C. and 290° C.

The vaporizer cartridge 17 and/or the base part 16 advantageously comprises a digital data memory 35 for storing information or parameters relating to the vaporizer cartridge 17. The data memory 35 can be part of, or connected to, the electronic control device 15. Advantageously, information on the composition of the liquid stored in the liquid reservoir 18; information on the process profile-in particular, power/temperature control; data for condition monitoring or system testing, e.g., leak testing; data relating to copy protection and protection against forgery; an ID for unambiguous identification of the vaporizer cartridge 17, serial number, production date and/or expiry date, and/or number of puffs (number of inhalations by the consumer) or of the time of use, are stored in the data memory 35.

The vaporization device 20 can, advantageously, have a measuring circuit 19 for determining the temperature of the heating element 21 by measuring the resistance of the heating element 21.

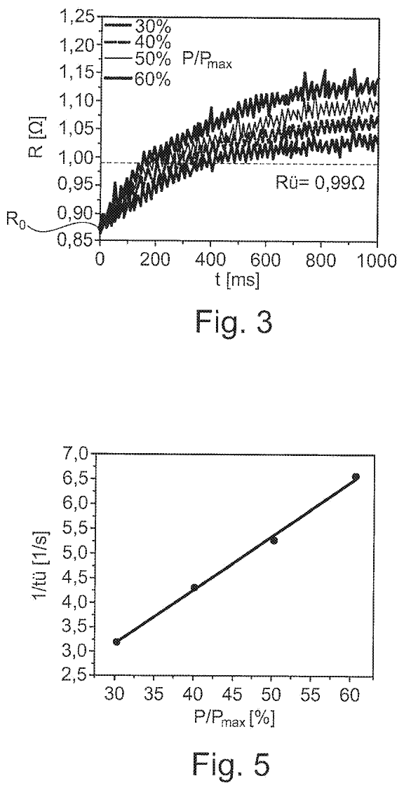

The diagrams in FIGS. 3 through 5 illustrate an initialization procedure according to the invention.

An initialization procedure is automatically started by the control device 15 when one or more predetermined initialization conditions are fulfilled and detected by the control device 15. Such an initialization condition is in particular a replacement of the cartridge, i.e., an initialization procedure is, advantageously, started after a vaporizer cartridge 17 has been inserted into the inhaler 10. The control device 15 is configured to detect such a replacement of the cartridge. This can take place, for example, via a detection circuit which continuously monitors and marks, by means of a digital value (flag), the presence or absence of a vaporizer cartridge 17 in a corresponding receptacle of the inhaler 10. A change in the digital value (flag) can be used, for example, as an interrupt for the software in the control device 15, so that it can, advantageously, be monitored at any time, e.g., also in standby mode, whether a vaporizer cartridge 17 is inserted in the inhaler 10 or not. The replacement of a cartridge can thus, advantageously, be detected in all operating states of the inhaler 10.

Additionally or alternatively, an initialization procedure can be started upon the detection of other events, e.g., the switching-on of the inhaler 10, and/or can be started in a time-controlled manner.

If the control device 15 detects an initialization condition, e.g., the insertion of a vaporizer cartridge 17 into the inhaler, the initialization procedure is started, and an inhalation request is output for this purpose to the consumer or user. This can take place via an optical, acoustic, and/or haptic display device of the inhaler 10. Additionally or alternatively, the inhalation request can be output to the user via an external electronic device which is connected to the inhaler 10 in a wireless or wired way.

If the control device 15 determines by means of the sensor (pressure sensor, pressure or flow switch) already mentioned that the user follows the inhalation request and puffs at the mouth end of the inhaler 10, the heating resistance 21 of the vaporizer 23 is operated by the consumer's (initialization) puff with a heating power P which is significantly lower than the maximum power Pmax during normal or consumption operation. The heating power P is set, for example, by means of pulse width modulation by the control device 15 in the heat current source 22.

In the exemplary embodiment according to FIGS. 3 through 5, four measurements corresponding to P/Pmax=30%, 40%, 50%, and 60% are considered, each measurement corresponding to at least one (initialization) puff by the consumer. The initialization procedure can take place on the basis of one or more puffs at a specific heating power (for example, at P/Pmax=40%) or comprise a plurality of measurements, via one or more puffs in each case, at different heating powers, whereby the accuracy of the initialization procedure can if necessary be increased. The low heating power P set in the initialization phase typically generates a quantity of vapor or an amount of active substance in the vapor that is too low is to be enjoyable or effective. The initialization phase lies timewise before the start of the actual consumption phase.

In FIG. 3, the (temperature-dependent) resistance R of the heating resistance 21 in ohms is plotted against the time in ms. The initial time t=0 ms corresponds to the start of the heating phase with low heating power P. The resistance curves R(t) for the first second (1,000 ms) of an initialization puff are shown with a relative power P/Pmax (or pulse width ratio) of 30%, 40%, 50%, and 60%. Here, the superimposed periodic signal is generated by pulse width modulation. The moving averages are plotted as thick lines against n measuring points (for example, n=31).

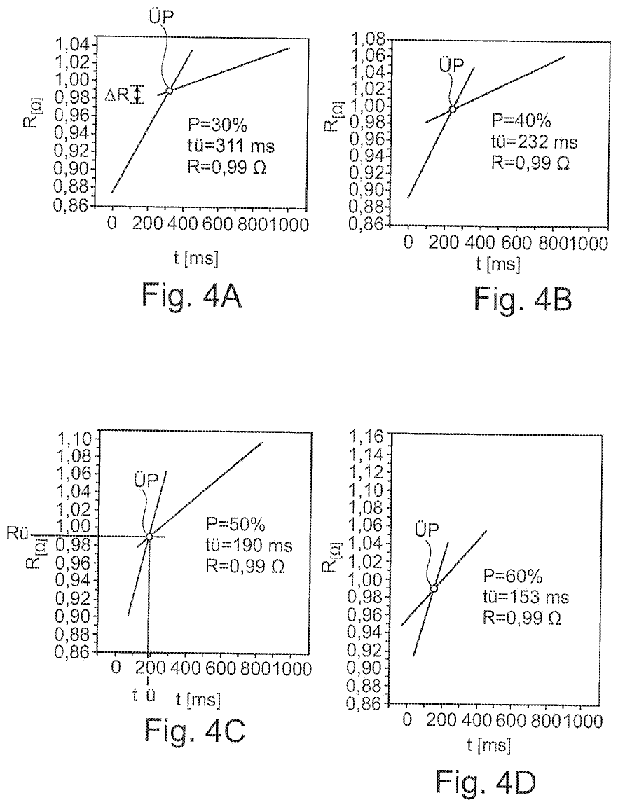

For better clarity, the four averaged curves (thick lines in FIG. 3) are shown also separately in FIGS. 4A through 4D.

As can be seen from FIGS. 3, 4A-4D, the temperature-dependent resistance starting from t=0 ms initially increases approximately linearly, with a relatively large slope, which is due to rapid heating of the heating element 21. In a later time range from 200 ms to 300 ms, the resistance increases further, but with a significantly smaller slope, which is due to a significantly slower heating of the heating element 21, because considerable vaporization starts from a certain point (transition point), and the vaporization energy is not available for heating the heating element 21.

The transition point UP (or vaporization point) between the region of low vaporization and the region of high vaporization (see FIGS. 4A-4D) constitutes a useful parameter for vaporization control in normal consumption operation of the inhaler 10. The control device 25 is therefore advantageously configured for calculating the transition point ÜP based upon the resistance-time-measurement series.

In one embodiment, the transition point ÜP is determined as an intersection of two straight lines, as shown in FIGS. 4A-4D. The first straight line is adapted or fitted to the measurement series in a region starting from t0=0 ms up to, for example, t1=100 ms or 200 ms, such that the deviation from the measurement curve or the smoothed measurement curve is minimal (region of low vaporization)-for example, to the first 50 measuring points at intervals of 2 ms each. The second straight line is adapted or fitted to the measurement series in a region starting from t2>t1 up to, for example, t3=600 ms, such that the deviation from the measurement curve or the smoothed measurement curve is again minimal (region of high vaporization). In all cases, i.e., regardless of the respective heating power P, a transition resistance (y-value of the transition point in the resistance-time diagrams) of Rü=0.99 Ω results. The heating duration or transition time tü (y-value of the transition point in the resistance-time diagrams), on the other hand, is different depending upon the heating power, because a higher heating power means faster heating and earlier reaching of the transition point (for example, P/Pmax=30%, tü=311 ms; P/Pmax=60%, tü=153 ms).

Other methods for determining the transition point from the measurement series are possible. For example, it could be sufficient to adapt or fit only one straight line to the measurement series in a region starting from t0=0 ms up to, for example, t1=100 ms or 200 ms, such that the deviation from the measurement curve or the smoothed measurement curve is minimal (region of low vaporization). The transition point ÜP is reached when the difference ΔR between the straight line and the corresponding measured resistance value (or the measurement curve or measurement series) exceeds a preset threshold value Rt; see FIG. 4A.

Further methods for determining the transition point ÜP are known from DE 10 2019 113 645.8, the disclosure of which is hereby incorporated into the present application. The determination of the transition point UP by means of an algorithm obtained using artificial intelligence methods is also possible, as has already been explained above.

In the diagram shown in FIG. 5, the inverse heating times 1/tü taken from FIGS. 4A-4D are plotted against the heating power P (here, relative to Pmax). The inverse heating times 1/tü lie on a straight line extending through the origin, because the applied heating energy is the same in all cases (regardless of the heating power P) until the transition point ÜP is reached.

The vaporizer 23 or the heating element 21 can be described by the following parameters: Rü—here, for example, 0.99 Ω; R0(T0), where T0 is the ambient temperature at start time t0=0 ms; here, for example, R0(T0)=0.88 Ω—see FIG. 3; and the slope of the curve in the diagram 1/tü vs. P; see FIG. 5. This parameter set, Rü, R0(T0), 1/tü slope, can be referred to as the model of the heater or vaporizer 23, or heater model for short.

In principle, a measurement at a specific heating power is sufficient to determine the transition point ÜP or all parameters of the heater model. However, the performance of several measurements at the same heating power and/or different heating powers increases the reliability and accuracy of the measurement.

The heater model (Rü, R0(T0), 1/tü slope) or individual parameters thereof are useful parameters for the vaporization control in a consumption phase following initialization. This control can be carried out, by way of example, as described in DE 10 2019 113 645.8, the disclosure of which relates to the current control of the vaporizer 23 in the present application. In the simplest case, the current value Iv of the referenced application can be determined from the transition resistance Rü: Iv=Rü/U, with U being the known heating voltage. Then, a control of the heating current Ih in a range I1<Iv<I2 can take place. Other vaporization controls based upon one or more parameters of the heater model and/or upon one or more parameters derived therefrom, such as tü, to, temperature at the transition point Tü, etc., are possible.

In light of the above, the control device 15 thus monitors one or more parameters during the following puffs in the consumption phase and initiates suitable measures in the case of values that differ considerably (from corresponding, stored target values).

### EMBODIMENTS

Embodiment 1. Method for determining at least one parameter for vaporization in an inhaler (10) comprising a vaporizer (23), based upon resistance heating, and an electronic control device (15), characterized in that the electronic control device (15) is configured to carry out the following initialization procedure—in particular, after a replacement of a vaporizer cartridge (17) in the inhaler (10):
outputting an inhalation request to a user of the inhaler (10);
in the case of a puff taken by the user following the inhalation request:
operating the vaporizer (23) with a comparatively low vaporization capacity P, and
recording a time measurement series of an electrical parameter of the vaporizer (23);
determining a transition point ÜP between a region of low vaporization and a region of high vaporization in the recorded time measurement series;
determining and storing at least one parameter associated with the initialization procedure.

Embodiment 2. Method according to embodiment 1, characterized in that the heating power P set during the initialization procedure is not more than 80% of the average heating power <P> or maximum heating power Pmax used in normal consumption operation.

Embodiment 3. Method according to one of the preceding embodiments, characterized in that one or more of the following parameters are determined and stored:
the value R0 of the electrical parameter at the beginning of the time measurement series at ambient temperature T0;
the ambient temperature T0;
the time period tü from an initial point of the time measurement series until the transition point ÜP is reached;
the value Rü of the electrical parameter at the transition point ÜP.

Embodiment 4. Method according to one of the preceding embodiments, characterized in that the electronic control device (15) calculates a measure of the quality of the determination of the transition point ÜP, and, in case of insufficient quality, causes the initialization procedure to be carried out again.

Embodiment 5. Method according to one of the preceding embodiments, characterized in that the heater of the vaporizer (23) is controlled on the basis of the at least one stored parameter after completion of the initialization procedure.

Embodiment 6. Method according to one of the preceding embodiments, characterized in that the initialization procedure is carried out successively at different heating powers of the vaporizer (23).

Embodiment 7. Method according to one of the preceding embodiments, characterized in that at least one of the determined and stored parameters is determined after a consumer puff in a checking procedure and is compared with a target value.

Embodiment 8. Method according to embodiment 7, characterized in that, in the case of deviations of the redetermined parameter from the target value, a predetermined measure is initiated-for example, performing a re-initialization procedure or outputting a message to the user.

Embodiment 9. Method according to one of the preceding embodiments, characterized in that the initialization procedure and/or the checking procedure is carried out after determination of at least one predetermined event, for example
replacement of a vaporizer cartridge (17);
switching on of the inhaler (10);
interruption of use of the inhaler (10) for a predetermined period of time;
change in ambient temperature T0 by a predetermined value.

Embodiment 10. Method according to one of the preceding embodiments, characterized in that an assessment by the consumer is requested in the initialization procedure and is, advantageously, taken into account when determining the at least one parameter.

Embodiment 11. Method according to one of the preceding embodiments, characterized in that the initialization procedure and/or the checking procedure is carried out
in a time-controlled manner or periodically, and/or
via one user puff or via a plurality of user puffs.

Embodiment 12. Method according to one of the preceding embodiments, characterized in that, when inserting a vaporizer cartridge (17) with an individual identifier and having the identifier read out by the electronic control device (15), at least one stored parameter assigned to this identifier can be accessed by the electronic control device (15).

Embodiment 13. Method according to one of the preceding embodiments, characterized in that the transition point ÜP is determined based upon a regression—preferably a linear regression-to the time measurement series.

Embodiment 14. Method according to one of the preceding embodiments, characterized in that the transition point ÜP is determined by means of an algorithm obtained using artificial intelligence methods.

Embodiment 15. Inhaler (10) comprising a vaporizer (23), based upon resistance heating, and an electronic control device (15), characterized in that the electronic control device (15) is configured to carry out the method according to one of the preceding embodiments.

1. A method for determining at least one parameter for vaporization in an inhaler, comprising:
providing an inhaler comprising a vaporizer, based upon resistance heating that comprises an electrical resistance, and an electronic control device;
carrying out the following initialization procedure via the electronic control device:
in the case of a puff taken by a user of the inhaler following the inhalation
operating the vaporizer with a comparatively low power P; and
recording a time measurement series of an electrical parameter of the vaporizer;
determining a transition point ÜP between a region of low vaporization and a region of high vaporization in the recorded time measurement series; and
determining and storing at least one parameter associated with the initialization procedure.

2. The method according to claim 1, wherein the heating-power P set during the initialization procedure is not more than 80% of an average power <P> or maximum-heating power Pmax used in normal consumption operation.

3. The method according to claim 1,
wherein the at least one parameter determined and stored comprises:
a value R0 of the electrical parameter at the beginning of the time measurement series at ambient temperature T0;
an ambient temperature T0;
a time period tü from an initial point of the time measurement series until the transition point ÜP is reached;
a value Rü of the electrical parameter of the vaporizer at the transition point ÜP.

4. The method according to claim 1, wherein the electronic control device calculates a measure of a quality of the determination of the transition point ÜP, and, in case of insufficient quality, causes the initialization procedure to be carried out again.

5. The method according to claim 1, wherein a heater of the vaporizer is controlled on the basis of the at least one stored parameter after completion of the initialization procedure.

6. The method according to claim 1, wherein the initialization procedure is carried out successively at different heating-powers of the vaporizer.

7. The method according to claim 1, wherein one or more of the at least one determined and stored parameter is redetermined after a puff taken by the user in a checking procedure and is compared with a target value.

8. The method according to claim 7, wherein in a case of deviations of the redetermined parameter from the target value, a predetermined measure is initiated.

9. The method according to claim 7,
wherein the initialization procedure and/or the checking procedure is carried out after determination of at least one predetermined event,
wherein the at least one predetermined event comprises one or more of the following:
replacement of a vaporizer cartridge;
switching on of the inhaler;
interruption of use of the inhaler for a predetermined period of time;
change in ambient temperature T0 by a predetermined value.

10. The method according to claim 1, wherein an assessment by the user is requested in the initialization procedure and is taken into account when determining the at least one parameter.

11. The method according to claim 7,
wherein the initialization procedure and/or the checking procedure is carried out:
in a time-controlled manner or periodically, and/or
via one user puff or via a plurality of user puffs.

12. The method according to claim 1, wherein, when inserting a vaporizer cartridge with an individual identifier and having the identifier read out by the electronic control device, at least one stored parameter assigned to this individual identifier can be accessed by the electronic control device.

13. The method according to claim 1, wherein the transition point ÜP is determined based upon a regression to the time measurement series.

14. The method according to claim 1, wherein the transition point ÜP is determined via an algorithm obtained using artificial intelligence methods.

15. An inhaler, comprising:
a vaporizer, comprising an electrical resistance, and
an electronic control device,
wherein the electronic control device is configured to carry out the method according to claim 1.

16. The method according to claim 1,
wherein the initialization procedure is carried out after replacing a vaporizer cartridge in the inhaler.

17. The method according to claim 8,
wherein the predetermined measure comprises performing a re-initialization procedure or outputting a message to the user.

18. The method according to claim 13,
wherein the regression is a linear regression.
